(19) United States
(12) Patent Application Publication
(10) Pub. No.: US 2004/0213826 A1
(43) Pub. Date: Oct. 28, 2004

(54) Medical devices and methods for inhibiting proliferation of smooth muscle cells

(76) Inventors: Steven O. Marx, New York, NY (US); Andrew Robert Marks, Larchmont, NY (US)

Correspondence Address: Leslie Gladstone Restaino, Esq., Brown Raysman Millstein Felder & Steiner LLP, 163 Madison Avenue, P.O. Box 1989, Morristown, NJ, 07962-1989, US

(21) Appl. No.: 10/425,354

(22) Filed: Apr. 28, 2003

(51) Int. Cl. A61K 38/12; A61K 31/19

(52) U.S. Cl. 424/423; 514/9; 514/575

(57) ABSTRACT

The present invention provides HDAC inhibitors for use in inhibiting proliferation and/or migration of smooth muscle cells. The present invention further provides medical devices coated with the HDAC inhibitors. The present invention also provides use of the medical devices in methods for inhibiting proliferation and/or migration of smooth muscle cells. Additionally, the present invention provides methods for inhibiting proliferation and/or migration of non-neoplastic smooth muscle cells. Finally, the present invention provides methods for preventing or treating restenosis after angioplasty or stent implantation in a subject.

### BACKGROUND OF THE INVENTION

Stenosis and restenosis are conditions associated with a narrowing of blood vessels. Stenosis of blood vessels generally occurs gradually over time. Restenosis, in contrast, relates to a narrowing of blood vessels following an endovascular procedure, such as balloon angioplasty and/or stent implantation, or a vascular injury. Stents are tiny mesh tubes, implanted into a blood vessel, that serve as scaffolding to prevent the vessel from becoming blocked.

Balloon angioplasty is typically performed to open a stenotic blood vessel; stenting is usually performed to maintain the patency of a blood vessel after, or in combination with, balloon angioplasty. A stenotic blood vessel is opened with balloon angioplasty by navigating a balloon-tipped catheter to the site of stenosis, and expanding the balloon tip effectively to dilate the occluded blood vessel. In an effort to maintain the patency of the dilated blood vessel, a stent may be implanted in the blood vessel to provide intravascular support to the opened section of the blood vessel, thereby limiting the extent to which the blood vessel will return to its occluded state after release of the balloon catheter. It is estimated that restenosis after balloon angioplasty and stent implantation occurs in over 33% of patients, which reduces the overall success of the relatively non-invasive balloon angioplasty and stenting procedures (Gruntzig, A., Transluminal dilatation of coronary-artery stenosis, Lancet, 1:263, 1978; Gruntzig et al., Nonoperative dilatation of coronary-artery stenosis: percutaneous transluminal coronary angioplasty. N. Engl. J. Med., 301:61-68, 1979; Bourassa et al., Report of the Joint ISFC/WHO Task Force on Coronary Angioplasty: The International Society and Federation of Cardiology and the World Health Organization. Circulation, 78:780-89, 1988; Bourassa et al., Long term follow-up of coronary angioplasty: the 1977-1981 National Heart, Lung and Blood Institute registry. Eur. Heart J., 10:36-41 (Supp. G), 1989; Poon et al., Overcoming restenosis with sirolimus: from alphabet soup to clinical reality. Lancet, 359:619-22, 2002).

Restenosis is attributed to many factors, including proliferation of smooth muscle cells (SMC). SMC proliferation is triggered by the initial mechanical injury to the intima that is sustained at the time of balloon angioplasty and stent implantation. The process is characterized by early platelet activation and thrombus formation, followed by SMC recruitment and migration, and, finally, cellular proliferation and extracellular matrix accumulation (Clowes et al., Kinetics of cellular proliferation after arterial injury. Lab. Invest., 49:327-33, 1983). Damaged endothelial cells, SMCs, platelets, and macrophages secrete cytokines and growth factors which promote the restenosis (Ip et al., The role of platelets, thrombin and hyperplasia in restenosis after coronary angioplasty. J.A.C.C., 77B-88B, 1991). Strategies targeted at the triggers of cellular growth have been evaluated, and most have unfortunately failed to adequately reduce restenosis after percutaneous transluminal coronary angioplasty (PTCA) and stent implantation, which is likely due to redundant signaling pathways (Marx et al., Bench to bedside: The development of rapamycin and its application to stent restenosis. Circulation, 104:852-55, 2001). SMC proliferation represents the final common pathway leading to neointimal hyperplasia. Therefore, anti-proliferative therapies aimed at inhibiting specific regulatory events in the cell cycle may constitute the most reasonable approach to restenosis after angioplasty (Marx et al., supra).

Recently, sirolimus (rapamycin) and paclitaxel (taxol) have been used in conjunction with balloon angioplasty and stenting, in an effort to limit the occurrence of restenosis after balloon angioplasty and stenting (Marx et al., supra). The rapamycin and taxol are delivered to the site susceptible to restenosis via stents that have been coated with these agents. Stents coated with rapamycin and taxol are described in U.S. Patent application Ser. No. 2002/0055206, entitled, “Antiproliferative drug and delivery device”, U.S. Pat. No. 6,159,142, entitled, “Stent with radioactive coating for treating blood vessels to prevent restenosis”, and U.S. Pat. No. 5,788,979, entitled “Biodegradable coating with inhibitory properties for applications to biocompatible materials”, each of which is hereby incorporated by reference. Although rapamycin- and taxol-coated stents have generated favorable results in reducing in-stent restenosis (Marx et al., supra), restenosis after angioplasty and/or stent implantation remains a significant medical problem (Poon et al., supra). Therefore, there exists a need for new agents that are effective in inhibiting, preventing, and/or treating restenosis.

Histones are proteins that are found in the nuclei of all eukaryotic cells, and complexed to DNA in chromatin and chromosomes. Histone deacetylase (HDAC) is an enzyme that catalyzes the deacetylation of histones. Specifically, HDAC hydrolyzes n-acetyl groups on histones. The blocking of HDAC activity by specific HDAC inhibitors, including trichostatin-A (TSA) and trapoxin (TPX), modulates the differentiation of normal and malignant cells. HDAC inhibitors have also been shown to inhibit cellular growth, to inhibit migration of certain cell types, to inhibit interleukin 2 (IL-2) gene expression, and to cause immunosuppression in a mouse model. Several structural classes of HDAC inhibitors have been identified, including the following: (1) short-chain fatty acids (butyrates); (2) hydroxamic acids (TSA, SAHA, and oxamflatin); (3) cyclic tetrapeptides (trapoxin A); (4) cyclic peptides such as FR901228 and apicidin; and (5) benzamides (MS-27-275).

Histone acetylation/deacetylation has been proposed to play a critical role in nucleic acid transcription (Allfrey, V. G., Structural modifications of histones and their possible role in the regulation of ribonucleic acid synthesis, Proc. Can. Cancer. Conf, 6:313-15, 1966). Core histones are acetylated at amino-terminal lysine residues, which causes a decrease in affinity for DNA (Wolffe, A. P., Histone deacetylase: a regulator of transcription, Science, 272:371-72, 1996). This process is predicted to increase the ability of transcriptional regulators to access regulatory regions. Deacetylation is believed to increase the strength of the histone/DNA interaction, and to decrease the access of transcription complexes to localized regions of DNA (Struhl, K., Histone acetylation and transcriptional regulatory mechanisms, Genes Dev., 12:599-606, 1998). However, prior to the present invention, it was not known to treat restenosis after angioplasty and/or stent implantation using stents coated with HDAC inhibitors.

### SUMMARY OF THE INVENTION

The inventors have developed stents coated with HDAC inhibitors, to inhibit the restenotic process. The HDAC inhibitor agents are hydrophobic, and inhibit tumor-cell growth in the nM range. It is believed that HDAC inhibitors rely upon a biological pathway that differs from those of other drugs currently used to coat stents. Therefore, the present invention provides HDAC inhibitors and medical devices, and methods for their use, that inhibit the proliferation of smooth muscle cells and prevent and/or treat conditions associated with proliferation of smooth muscle cells.

Accordingly, in one aspect of the present invention, a medical device is provided that has a coating which includes an effective amount of HDAC inhibitor. The medical device coated with the HDAC inhibitor is generally capable of inhibiting proliferation and/or migration of smooth muscle cells in a subject's vasculature, which may result from use of the medical device. A variety of HDAC inhibitors may be used in the medical device of the present invention, such as trichostatin-A, suberoylanilide hydroxamic acid (SAHA), trapoxin, butyric acid, MS-27-275, oxamflatin, apicidin, depsipeptide, and depudecin. The medical device may be a coated balloon catheter, which may inhibit proliferation of smooth muscle cells that results from a balloon angioplasty. The medical device also may be a coated stent, which may inhibit proliferation of smooth muscle cells that results from stent implantation in a subject's vasculature.

In one embodiment of the present invention, the coating on the medical device includes a biodegradable carrier that degrades over time, such that the HDAC inhibitor is eluted from the medical device over time. The coating may also include a non-thrombogenic agent that is also eluted from the medical device as the biodegradable carrier degrades. In another embodiment, the medical device includes a plurality of coatings, each of which includes a biodegradable carrier and at least one HDAC inhibitor, thereby providing staged release of the HDAC inhibitor(s) from the medical device. In a further embodiment, at least one of the plurality of coatings includes an active ingredient, such that the active ingredient is eluted from the medical device by timed release.

In another aspect of the present invention, use of a medical device is provided in a method for inhibiting proliferation and/or migration of smooth muscle cells, wherein the medical device has a coating comprising an HDAC inhibitor.

In another aspect of the present invention, a medical device is provided which has a coating that includes an effective amount of an HDAC inhibitor agent and a biodegradable carrier that degrades over time such that the HDAC inhibitor is eluted from the stent over time, wherein the HDAC inhibitor is selected from the group consisting of trichostatin-A, suberoylanilide hydroxamic acid (SAHA), trapoxin, butyric acid, MS-27-275, oxamflatin, apicidin, depsipeptide, and depudecin.

In another aspect of the present invention, use of a medical device is provided in a method for inhibiting proliferation and/or migration of smooth muscle cells, wherein the medical device has a coating that includes an effective amount of an HDAC inhibitor agent and a biodegradable carrier that degrades over time such that the HDAC inhibitor is eluted from the stent over time, and wherein the HDAC inhibitor is selected from the group consisting of trichostatin-A, suberoylanilide hydroxamic acid (SAHA), trapoxin, butyric acid, MS-27-275, oxamflatin, apicidin, depsipeptide, and depudecin.

In another aspect of the present invention, a stent for implantation in a blood vessel is provided, wherein the stent has a coating which includes an effective amount of an HDAC inhibitor and a biodegradable carrier that degrades over time such that the HDAC inhibitor is eluted from the stent over time, and wherein the HDAC inhibitor is selected from the group consisting of trichostatin-A, suberoylanilide hydroxamic acid (SAHA), trapoxin, butyric acid, MS-27-275, oxamflatin, apicidin, depsipeptide, and depudecin.

In another aspect of the present invention, a method is provided for inhibiting proliferation and/or migration of non-neoplastic smooth muscle cells and/or for preventing, treating, or inhibiting the occurrence of a condition associated with proliferation of non-neoplastic smooth muscle cells in a subject, by administering to the subject an amount of an HDAC inhibitor effective to inhibit proliferation of smooth muscle cells in the subject. A variety of HDAC inhibitors may be used in the method of the present invention, including, without limitation, trichostatin-A, suberoylanilide hydroxamic acid (SAHA), trapoxin, butyric acid, MS-27-275, oxamflatin, apicidin, depsipeptide, and depudecin. Examples of conditions associated with proliferation of non-neoplastic smooth muscle cells, which may be prevented or treated by the method of the present invention, include, without limitation, stenosis, restenosis after angioplasty, restenosis after stent implantation, and accelerated arteriopathy after cardiac transplantation.

It is to be understood that the step of administering an HDAC inhibitor to a subject may be accomplished by a variety of methods or procedures. In one embodiment of the present invention, the HDAC inhibitor is coated on a medical device, which may be administered directly to the site in the subject that is susceptible to proliferation of smooth muscle cells. The medical device may be a stent that is implanted into a subject's vasculature, to maintain the patency of the subject's blood vessel, as may be required in connection with a balloon angioplasty procedure.

In another aspect of the present invention, a method is provided for preventing, treating, or inhibiting the occurrence of restenosis after angioplasty or stent implantation in a subject in need, by administering to the subject an amount of an HDAC inhibitor effective to prevent restenosis. In one embodiment, the HDAC inhibitor is trichostatin-A. The trichostatin-A may be coated on a stent, and administered directly to the subject, at a site susceptible to proliferation of smooth muscle cells, by implanting the stent into the subject's vasculature.

Additional aspects of the present invention will be apparent in view of the description which follows.

### BRIEF DESCRIPTION OF THE FIGURES

FIG. 1 is a bar graph that sets forth the effects of rapamycin and trichostatin-A on rat aortic smooth muscle cell proliferation at various concentrations.

FIG. 2 is a bar graph that sets forth the effect of oxamflatin on rat aortic smooth muscle cell proliferation at various concentrations.

FIG. 3 is a bar graph that sets forth the effect of trichostatin-A on human aortic smooth muscle cell proliferation at various concentrations.

### DETAILED DESCRIPTION OF THE INVENTION

The present invention relates to medical devices and methods that may be used to inhibit smooth muscle cell (SMC) proliferation, and thereby to prevent, treat, or inhibit the occurrence of conditions associated with SMC (particularly vascular SMC) proliferation, such as stenosis, restenosis, and transplant arteriopathy. The medical devices and methods of the present invention utilize inhibitors of histone deacetylase (HDAC).

Histone acetylation/deacetylation has been proposed to play a critical role in gene expression regulation (Allfrey, V. G., Structural modifications of histones and their possible role in the regulation of ribonucleic acid synthesis, Proc. Can. Cancer. Conf., 6:313-15, 1966). Core histones are acetylated at amino-terninal lysine residues, which causes a decrease in affinity for DNA (Wolffe, A. P., Histone deacetylase: a regulator of transcription, Science, 272:371-72, 1996). It is predicted that this process will increase the ability of transcriptional regulators to access regulatory regions. Deacetylation is believed to increase the histone/DNA interaction, and decrease the access of transcription complexes to localized regions of DNA (Struhl, K., Histone acetylation and transcriptional regulatory mechanisms, Genes Dev., 12:599-606, 1998). The blocking of histone deacetylase (HDAC) activity by specific inhibitors, including trichostatin-A (TSA), oxamflatin, suberoylanilide hydroxamic acid (SAHA), and trapoxin, modulates the differentiation of some normal and malignant cells (Yoshida et al., Potent and specific inhibition of mammalian histone deacetylase both in vivo and in vitro by trichostatin A, J. Biol. Chem., 265:17174-179, 1990; Richon et al., A class of hybrid polar inducers of transformed cell differentiation inhibits histone deacetylase, Proc. Nat'l Acad. Sci., 95:3003-07, 1998; Kijima et al., Trapoxin, an antitumor cyclic tetrapeptide, is an irreversible inhibitor of mammalian histone deacetylase, J. Biol. Chem., 268:22429-435,1993; Deroanne et al., Histone deacetylase inhibitors as anti-angiogenic agents altering vascular endothelial growth factor signaling, Oncogene, 21:427-36, 2002; Marks et al., Histone deacetylase inhibitors: inducers of differentiation or apoptosis of transformed cells, J. Nat'l Cancer Inst., 92:1210-16, 2000; Kim et al., Oxamflatin is a novel antitumor compound that inhibits mammalian histone deacetylase, Oncogene, 18:2461-70, 1999).

Since it is known that histone acetylation/deacetylation regulates transcription through a chain of events involving acetylation of core histones at amino-terminal lysine residues, followed by a decrease in affinity for DNA and an increase in the ability of transcriptional regulators to access regulatory regions, it is believed that inhibition of HDAC activity may modulate gene expression in cells.

A variety of compounds are known to inhibit HDAC activity, including, without limitation, trichostatin-A, suberoylanilide hydroxamic acid (SAHA), trapoxin, butyric acid, MS-27-275, oxamflatin, apicidin, depsipeptide, and depudecin (Yoshida et al., supra; Richon et al., supra; Kijima M et al., supra; Deroanne et al., supra; Marks et al., supra). HDAC inhibitor compounds may be divided into a variety of structural classes of compounds, including, without limitation, short-chain fatty acids (e.g., butyrates), hydroxamic acids (e.g., TSA, SAHA, and oxamflatin), cyclic tetrapeptides (e.g., trapoxin), cyclic peptides (e.g., FR901228 and apicidin), and benzamides (e.g., MS-27-275).

HDAC inhibitors have been shown to inhibit proliferation of transformed cells in culture, and some HDAC inhibitors have been shown to inhibit tumor growth in animal models (Marks et al., supra). The butyrates class of HDAC inhibitor compounds is approved for clinical use; however, they are not ideal candidates with respect to inhibition of cell proliferation, primarily because of the high concentrations (millimolar) necessary to inhibit HDAC activity (Marks et al., supra).

Trichostatin-A (TSA) has been shown to be a potent inducer of murine erythroleukemia cell differentiation, and a specific inhibitor of the mammalian cell cycle, blocking cell-cycle progression at both G1 and G2 phases (Hoshikawa et al., Trichostatin A induces morphological changes and gelsolin expression by inhibiting histone deacetylase in human carcinoma cell lines, Experimental Cell Res., 214:189-97, 1994). TSA has also been shown to inhibit growth of certain cells, such as Balb/c-3T3 cells. It has been demonstrated that TSA increases p27^{kiP1} levels in 3T3 cells; however, this increase was deemed a secondary effect, since TSA is capable of inhibiting the growth of p27 and p21 null cells (Wharton et al., Inhibition of mitogenesis in Balb/c-3T3 cells by trichostatin A, J. Biol. Chem., 275:33981-987, 2000).

TSA (400 nM) and SAHA have been shown to prevent vascular-endothelial-growth-factor-stimulated human umbilical endothelial cells from invading a type I collagen gel, and from forming capillary like structures. TSA (10 nM) and SAHA (400 nM) were shown to inhibit angiogenesis in embryoid bodies (Deroanne et al., supra). TSA has also been shown to suppress collagen synthesis, to prevent TGF,-β1-induced fibrogenesis in skin fibroblasts (Rombouts et al., Trichostatin A, a histone deacetylase inhibitor, suppresses collagen synthesis and prevents TGF-beta(1)-induced fibrogenesis in skin fibroblasts, Experimental Cell Res., 278:184-97, 2002), and to decrease the expression of RhoA, a mediator in the development of the actin cytoskeleton, in hepatic stellate cells (Rombouts et al., Actin filament formation, reorganization and migration are impaired in hepatic stellate cells under influence of trichostatin A, a histone deacetylase inhibitor, J. Hepatol, 37:788-96, 2002). Additionally, one study has suggested that platelet-derived-growth-factor-(PDGF-) induced competence of primary cultured SMCs from rat thoracic aorta was inhibited by TSA (1 μg/ml) (Okabe et al., Competence effect of PDGF on Ki-67 antigen and DNA contents, and its inhibition by trichostatin-A and a butylydene phthalide BP-421 in primary smooth muscle cells of rat aorta by flow cytometry, Biol. Pharm. Bull., 18:1665-70, 1995).

HDAC inhibitors, however, have also been shown to down-regulate the expression of endothelial nitric oxide (NO) synthase. For example, TSA (1 mM) attenuated the NO-dependent relaxation of porcine coronary arteries (Rossig et al., Inhibitors of histone deacetylation down regulate the expression of endothelial nitric oxide synthase and compromise endothelial cell function in vasorelaxation and angiogenesis, Circ. Res., 91:837-44, 2002).

Accordingly, the inventors compared the effects of rapamycin and HDAC inhibitors on SMC proliferation. The data, which are presented below, suggest that inhibition of HDAC activity produces profound inhibition of SMC proliferation and/or migration. The inventors propose that HDAC inhibition provides a novel mechanism to prevent or limit the occurrence of vascular conditions associated with proliferation of non-neoplastic SMCs, such as stenosis, restenosis after angioplasty and/or stent implantation, and accelerated arteriopathy after cardiac transplantation. Additionally, the inventors' studies show that HDAC inhibitors can be potent inhibitors of vascular SMC proliferation at concentrations at or below those needed for rapamycin effectively to inhibit vascular SMC proliferation. Moreover, the inventors' data suggest that HDAC inhibitors, particularly TSA, inhibit extracellular matrix deposition, which is a step in the restenosis process.

Many HDAC inhibitor compounds are hybrid polar compounds, since they have in common two polar groups separated by an apolar 5-6 carbon methylene chain, and also exhibit limited solubility in water. TSA has a molecular weight of 302, and is soluble in DMSO and ethanol. With regard to immunosuppression, TSA (IC₅₀=73 nM) has been shown to inhibit IL-2 expression in Jurkat cells (Takahashi et al., Selective inhibition of IL-2 gene expression by trichostatin A, a potent inhibitor of mammalian histone deacetylase. J. Antibiot., 49:453-57, 1996 (Tokyo); Koyama et al., Histone deacetylase inhibitors suppress IL-2-mediated gene expression prior to induction of apoptosis, Blood, 96:1490-95, 2000; Nambiar et al., Effect of trichostatin A on human T cells resembles signaling abnormalities in T cells of patients with systemic lupus erythematosus: a new mechanism for TCR zeta chain deficiency and abnormal signaling, J. Cell Biochem., 85:459-69, 2002).

In summary, the HDAC inhibitors are soluble in DMSO and ethanol, are small, and are extremely potent anti-proliferative and immunosuppressive agents. Additionally, since HDAC inhibitors are not cytotoxic, they are likely to have little or no toxicity in the blood vessel walls. Furthermore since HDAC inhibitors inhibit inflammation, are immunosuppressant, and likely inhibit migration and extracellular matrix formation, HDAC inhibitors have biological properties that are ideally suited for inhibiting, preventing, and/or treating vascular SMC proliferative diseases.

In view of the foregoing, the present invention provides a medical device for use in inhibiting proliferation and/or migration of smooth muscle cells (e.g., vascular SMC), wherein the medical device has a coating comprising at least one HDAC inhibitor. The medical device may be particularly useful for preventing, treating, or inhibiting the occurrence of conditions associated with vascular SMC proliferation, including, without limitation, restenosis, restenosis after angioplasty and/or stent implantation, and accelerated arteriopathy after cardiac transplantation.

HDAC may be inhibited by disabling, disrupting, or inactivating the function or activity of HDAC, or by diminishing the amount or expression of HDAC in a cell or tissue. Furthermore, HDAC function or activity may be inhibited by targeting HDAC directly, or by targeting HDAC indirectly by directly or indirectly causing, inducing, or stimulating the down-regulation of HDAC activity or expression within a cell or tissue. As used herein, “an HDAC inhibitor” shall include a protein, polypeptide, peptide, nucleic acid (including DNA, RNA, and an antisense oligonucleotide), antibody (monoclonal and polyclonal, as described above), Fab fragment (as described above), F(ab′)² fragment, molecule, compound, antibiotic, drug, and any combinations thereof, and may be an agent reactive with HDAC (i.e., it has affinity for, binds to, or is directed against HDAC). Additionally, the HDAC inhibitor may be an oligonucleotide antisense to HDAC, or RNAi directed against a nucleic acid encoding HDAC.

The HDAC inhibitor of the present invention may be any known in the art, including any of those described above. In one embodiment of the present invention, the HDAC inhibitor is trichostatin-A, suberoylanilide hydroxamic acid (SAHA), trapoxin, butyric acid, MS-27-275, oxamflatin, apicidin, depsipeptide, or depudecin. In one embodiment, the HDAC inhibitor is trichostatin-A, oxamflatin, or SAHA. It is to be understood that a number of compounds or agents that are not listed herein also inhibit HDAC activity. Accordingly, the list of exemplary HDAC inhibitor compounds or agents set forth herein is not limited thereto.

In accordance with the device of the present invention, the HDAC inhibitor may be provided in an amount effective to inhibit proliferation of smooth muscle cells in a subject. The subject may be any animal, including amphibians, birds, fish, mammals, and marsupials, but is preferably a mammal (e.g., a human; a domestic animal, such as a cat, dog, monkey, mouse, and rat; or a commercial animal, such as a cow or pig). In a preferred embodiment, the subject is a human. An effective amount of an HDAC inhibitor compound generally refers to an amount and/or concentration of inhibitor necessary to achieve a desired result—in this case, inhibition of SMC proliferation and/or migration. Accordingly, it is also understood that the effective amount of HDAC inhibitor on the medical device may vary. For instance, the effective amount may vary depending upon the desired amount or degree of SMC-proliferation inhibition, the subject's weight, severity of the subject's condition, etc.

The medical device of the present invention may be used to inhibit proliferation of SMCs in a subject by introducing the medical device into the subject at a site susceptible to SMC proliferation. It is to be understood that the present invention may be used to limit SMC proliferation in a variety of venous and arterial blood vessels. It is also understood that the coated medical device may be designed for use in various types of medical procedures. The medical device is preferably introduced to the subject intravascularly; however, the device may also be introduced into the subject via open surgical intervention.

In one embodiment of the present invention, the medical device is a stent for implantation in a subject's blood vessel to maintain the patency of the vessel. The stent may be implanted in connection with an angioplasty procedure or in other instances or procedures that may trigger SMC proliferation, including, without limitation, vascular injury, graft implantation or transplantation, and cardiac transplantation. In another embodiment, the medical device of the present invention is a catheter, such as an angioplasty balloon catheter, which, when coated with at least one HDAC inhibitor, may inhibit SMC proliferation during the initial injury caused by opening the occluded blood vessel therewith. Catheters coated with HDAC inhibitors may also aid in preventing or treating SMC proliferation that results from injury to blood vessels arising from navigation of the catheter to a site in the subject where an intravascular intervention procedure will occur.

SMC proliferation may also be inhibited with a combination of coated medical devices, including a coated stent in combination with a coated balloon catheter. In this instance, the combination would provide HDAC inhibitor compounds at all stages of the angioplasty and stenting procedures. The devices may also be coated with non-thrombogenic or thrombolytic agents that inhibit the formation of, or that break up, a thrombus. An example of such an agent is heparin.

The medical device of the present invention may be manufactured from a variety and/or a combination of biocompatible and non-biocompatible materials, including, without limitation, polyester, Gortex, polytetrafluoroethyline (PTFE), polyethelene, polypropylene, polyurethane, silicon, steel, stainless steel, titanium, Nitinol or other shape memory alloys, copper, silver, gold, platinum, Kevlar fiber, and carbon fiber. Where non-biocompatible materials may come into contact with a subject's anatomy, the components made from the non-biocompatible materials may be covered or coated with a biocompatible material.

The medical device of the present invention also may be coated using a variety of techniques, including dipping, spraying, etc. In one embodiment, the medical device, particularly a stent, is coated with at least one biodegradable carrier, such as a degradable or erodeable polymer, which includes therein an effective amount of the HDAC inhibitor. The biodegradable carrier degrades over time, thereby allowing the HDAC inhibitor (or other compound or agent therein) to elute from the stent over time. The term “elute” is used herein to denote the release or separation of a compound or agent from the medical device, and, therefore, is not limited to any particular mechanism unless otherwise noted. The medical device may be coated with the biodegradable carrier in various thicknesses. Generally, the greater the thickness of the coating, the longer it will take for the inhibitor, compound, or agent therein to elute from the medical device. The preferred duration of therapy would range from 7 days to 2 months.

In one embodiment of the present invention, the medical device is coated with the biodegradable carrier in layers, with each coating or layer providing a different or additional active ingredient (e.g., another HDAC inhibitor, a non-thrombogenic agent, etc.), thereby providing timed release of the active ingredient. For example, the medical device may be coated with a first layer that consists of a biodegradable carrier, an HDAC inhibitor, and a non-thrombogenic agent, and a second layer that consists of a biodegradable carrier and the same or another HDAC inhibitor. In this instance, the non-thrombogenic agent may be eluted for a limited time (e.g., during degradation of the first layer), or in a timed-release manner. Additionally, this embodiment would permit elution of different types of active ingredients (e.g., HDAC inhibitors) at different times (e.g., a first HDAC inhibitor may be eluted during degradation of the first layer, and a second HDAC inhibitor may be eluted during degradation of the second layer), in a timed-release manner. In a preferred embodiment, the present invention provides a stent for implantation in a blood vessel, wherein the stent has a coating comprising a biodegradable carrier that degrades over time and an HDAC inhibitor, and wherein the HDAC inhibitor is trichostatin-A or oxamflatin.

In another embodiment of the present invention, different sides of the medical device may be coated in single or multiple layers with biodegradable carriers which include therein different active ingredients, thereby permitting staged release of the active ingredients. For instance, the exterior side of a medical device, such as a stent (e.g., the portion which, when implanted in a subject, contacts the subject's vasculature), may be coated with a biodegradable carrier which includes an HDAC inhibitor; the opposite side of the device, which is exposed to a subject's blood, then may be coated with a non-thrombogenic agent or a biodegradable carrier containing a non-thrombogenic agent. In a further embodiment, the medical includes therein structures, such as pores or other reservoir systems, which are capable of holding the HDAC inhibitor. In this instance, a suitable release mechanism, such as a membrane, may be used to release the HDAC inhibitor from the medical device.

In view of the foregoing, the present invention further provides a use of a medical device in a method for inhibiting proliferation and/or migration of smooth muscle cells, wherein the medical device has a coating comprising an HDAC inhibitor. Additionally, the present invention provides a use of a medical device in a method for inhibiting proliferation and/or migration of SMCs, wherein the medical device has a coating comprising a biodegradable carrier that degrades over time and an HDAC inhibitor, and wherein the HDAC inhibitor is selected from the group consisting of trichostatin-A, suberoylanilide hydroxamic acid (SAHA), trapoxin, butyric acid, MS-27-275, oxamflatin, apicidin, depsipeptide, and depudecin.

The present invention further provides a method for inhibiting proliferation and/or migration of non-neoplastic smooth muscle cells in a subject in need. As used herein, the term “inhibiting proliferation” means inhibiting cell division and cell growth, and includes limiting the proliferative rate of cells. Inhibition of the growth, proliferation, and migration of SMCs may be detected by known procedures, including any of the methods, molecular procedures, and assays disclosed herein. Additionally, as used herein, the term “non-neoplastic” refers' to SMCs that are not derived from a neoplasm. As further used herein, a “neoplasm” is any uncontrolled and progressive multiplication of tumor cells (including abnormal cells), or any new and abnormal growth, under conditions that would not elicit, or would cause cessation of, multiplication of normal cells. In one embodiment of the present invention, the non-neoplastic SMCs are vascular smooth muscle cells. The subject may be any of those described above. In a preferred embodiment, the subject is known, or believed, to be susceptible to a condition associated with proliferation of non-neoplastic SMCs.

In accordance with the method of the present invention, an HDAC inhibitor is administered to the subject in an amount effective to inhibit the proliferation of SMCs in the subject. The amount of modulator of HDAC inhibitor that is effective to inhibit the proliferation of SMCs in a subject will vary depending on the particular factors of each case, including the type of SMCs, the location of the SMCs, the subject's weight, the severity of the subject's condition, and the method of administration. These amounts can be readily determined by the skilled artisan. In one embodiment of the present invention, the proliferation of SMCs is associated with a vascular condition, such as stenosis, restenosis after angioplasty, restenosis after stent implantation, and accelerated arteriopathy after cardiac transplantation.

HDAC inhibitor compounds and agents may be administered to a subject by known procedures, including, without limitation, oral administration, parenteral administration (e.g., epifascial, intracapsular, intracutaneous, intradermal, intramuscular, intraorbital, intraperitoneal, intraspinal, intrasternal, intravascular, intravenous, parenchymatous, or subcutaneous administration), transdermal administration, administration by osmotic pump, and implantation, introduction, or insertion of an HDAC-inhibitor-coated medical device. For oral administration, the HDAC inhibitor may be presented as capsules, tablets, powders, granules, or as a suspension. The inhibitor may be formulated with conventional additives, such as lactose, mannitol, cornstarch, or potato starch. The formulation may also be presented with binders, such as crystalline cellulose, cellulose derivatives, acacia, cornstarch, or gelatins. Additionally, the formulation may be presented with disintegrators, such as cornstarch, potato starch, or sodium carboxymethylcellulose. The formulation also may be presented with dibasic calcium phosphate anhydrous or sodium starch glycolate. Finally, the formulation may be presented with lubricants, such as talc or magnesium stearate.

For parenteral administration, the HDAC inhibitor compound or agent may be combined with a sterile aqueous solution, which is preferably isotonic in relation to the blood of the subject. Such a formulation may be prepared by dissolving the HDAC inhibitor in water containing physiologically-compatible substances, such as sodium chloride, glycine, and the like, and having a buffered pH compatible with physiological conditions, so as to produce an aqueous solution, then rendering said solution sterile. The formulation may be presented in unit or multi-dose containers, such as sealed ampules or vials. The formulation may be delivered by any mode of injection, including any of those described above.

For transdermal administration, the HDAC inhibitor compound or agent may be combined with skin-penetration enhancers, such as propylene glycol, polyethylene glycol, isopropanol, ethanol, oleic acid, N-methylpyrrolidone, and the like, which increase the permeability of the skin to the inhibitor, thereby allowing it to penetrate through the skin and into the bloodstream. The inhibitor may be further combined with a polymeric substance, such as ethylcellulose, hydroxypropyl cellulose, ethylene/vinylacetate, polyvinyl pyrrolidone, and the like, to provide the composition in gel form, which may be dissolved in solvent, such as methylene chloride, evaporated to the desired viscosity, and then applied to backing material to provide a patch. The inhibitor may be administered transdermally, at or near the site on the subject where the intimal hyperplasia, or proliferation of SMCs, is localized or expected to arise. Alternatively, the inhibitor may be administered transdermally at a site other than the affected area, in order to achieve systemic administration.

The HDAC inhibitor compound or agent may also be released or delivered from an osmotic mini-pump or other time-release device. The release rate from an elementary osmotic mini-pump may be modulated with a microporous, fast-response gel disposed in the release orifice. An osmotic mini-pump would be useful for controlling release, or targeting delivery, of the HDAC inhibitor.

Additionally, the HDAC inhibitor compound or agent may be administered to a subject via a medical device (e.g., a stent) coated therewith, as described above. Such a device may be inserted, introduced, or implanted into a subject (e.g., the subject's vasculature), at or near the site on the subject where the intimal hyperplasia, or proliferation of SMCs, is localized or expected to arise, thereby allowing the HDAC inhibitor to elute from the device into the surrounding vasculature. As described herein, the medical device may be constructed such that the HDAC inhibitor and/or another active ingredient is eluted from the device in a staged-release or timed-release manner. In one embodiment of the present invention, the medical device is implanted into a subject's vasculature in connection with a balloon angioplasty procedure. In another embodiment, local therapy is achieved with nanospheres impregnated with the HDAC inhibitor (Chorny et al., Study of the drug release mechanism from tyrphostin AG-1295-loaded nanospheres by in situ and external sink methods. J. Controlled Release, 83:401-14, 2002).

It is also within the confines of the present invention that the HDAC inhibitor compound or agent may be further associated with a pharmaceutically-acceptable carrier, thereby comprising a pharmaceutical composition. Accordingly, the present invention further provides a pharmaceutical composition, comprising the HDAC inhibitor and a pharmaceutically-acceptable carrier. The pharmaceutically-acceptable carrier must be “acceptable” in the sense of being compatible with the other ingredients of the composition, and not deleterious to the recipient thereof. The pharmaceutically-acceptable carrier employed herein is selected from various organic or inorganic materials that are used as materials for pharmaceutical formulations, and which may be incorporated as analgesic agents, buffers, binders, disintegrants, diluents, emulsifiers, excipients, extenders, glidants, solubilizers, stabilizers, suspending agents, tonicity agents, vehicles, and viscosity-increasing agents. If necessary, pharmaceutical additives, such as antioxidants, aromatics, colorants, flavor-improving agents, preservatives, and sweeteners, may also be added. Examples of acceptable pharmaceutical carriers include carboxymethyl cellulose, crystalline cellulose, glycerin, gum arabic, lactose, magnesium stearate, methyl cellulose, powders, saline, sodium alginate, sucrose, starch, talc, and water, among others.

The pharmaceutical composition of the present invention may be prepared by methods well-known in the pharmaceutical arts. For example, the composition may be brought into association with a carrier or diluent, as a suspension or solution. Optionally, one or more accessory ingredients (e.g., buffers, flavoring agents, surface active agents, and the like) also may be added. The choice of carrier will depend upon the route of administration of the composition. Formulations of the composition may be conveniently presented in unit dosage, or in such dosage forms as aerosols, capsules, elixirs, emulsions, eye drops, injections, liquid drugs, pills, powders, granules, suppositories, suspensions, syrup, tablets, or troches, which can be administered by any of the modes of administration described above.

The present invention further provides a method for preventing or treating restenosis after angioplasty or stent implantation in a subject, by administering to the subject an amount of an HDAC inhibitor effective to prevent restenosis in the subject. The HDAC inhibitor may be any of those described above, including trichostatin-A, suberoylanilide hydroxamic acid (SAHA), trapoxin, butyric acid, MS-27-275, oxamflatin, apicidin, depsipeptide, and depudecin. In one embodiment of the present invention, the HDAC inhibitor is trichostatin-A. In a preferred embodiment of the invention, the trichostatin-A is coated on a stent, and is administered directly to the subject by implanting the stent into the subject at a site susceptible to restenosis.

The present invention is described in the following Examples, which are set forth to aid in an understanding of the invention, and should not be construed to limit in any way the scope of the invention as defined in the claims which follow thereafter.

### EXAMPLES

### Example 1

### Cell Cultures

Low-passage rat aortic smooth muscle cells (RASM cells) from primary isolates and human aortic smooth muscle cells (HASM cells) were obtained and grown according to the suppliers' instructions. RASM cells were cultured in a medium comprising DMEM+10% FBS+100 U/ml penicillin and 100 ng/ml streptomycin, which was changed every 48 h. Micro-cultures of 5000 cells of each of the RASM and HASM were established in quadruplicate, placedin flat-bottom 96-well micro-titer plates, and exposed to various concentrations of the trichostatin-A, oxamflatin, and rapamycin. Trichostatin-A and oxamflatin were dissolved in dimethyl sulfoxide (DMSO) at various concentrations, and stored at −20° C. prior to use; rapamycin was dissolved in methanol (100 nM). After 48 h following initial exposure, the cultures were pulsed with 20 ml of a cell-proliferation assay solution (Promega CellTiter 96 AQᵤₑₒᵤₛ One Solution, Catalog G3580), and incubated for 1 h. The cultures' light absorbance was then measured on a BioRad Benchmark micro-plate reader, which was set to the range Ab 490 nm-Ab 655 nm. Light absorbed by the blank was accounted for in the tests. All experiments were repeated at least twice. Cell viability was determined using trypan blue staining, after incubating RASM cells with 100 ng/ml of rapamycin and trichostatin-A for 48 h.

### Example 2

### Exposure of RASM Cells to Trichostatin-A

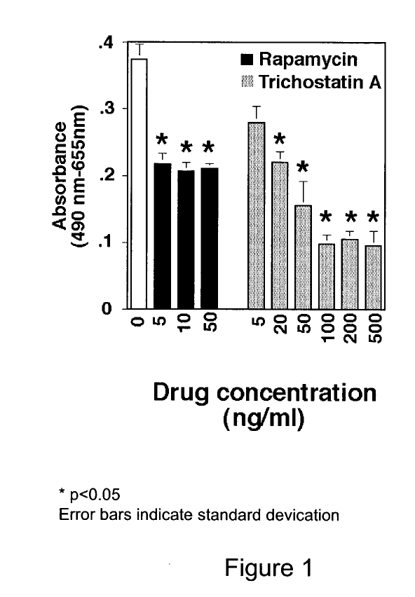

Referring to FIG. 1, micro-cultures consisting of RASM cells were plated into a 96-well dish, and exposed as follows: a number of the micro-cultures were sham exposed, a number were exposed to rapamycin (5, 10, and 50 ng/ml, respectively), and a number were exposed to trichostatin-A (5, 20, 50, 100, 200, and 200 ng/ml, respectively). The RASM cells were exposed for 48 h, prior to assessing cell proliferation.

### Example 3

### Exposure of HASM Cells to Trichostatin-A

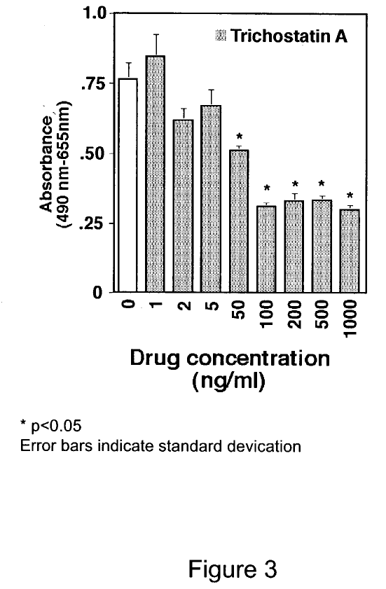

Referring to FIG. 3, micro-cultures of HASM cells were plated into a 96-well dish, and exposed as follows: a number of the micro-cultures were sham exposed, and a number were exposed to trichostatin-A (1, 2, 5, 50, 100, 200, 500, and 1000 ng/ml, respectively) concentrations. The HASM cells were exposed for 48 h before assessing cell proliferation.

### Example 4

### Exposure of RASM Cells to Osamflatin

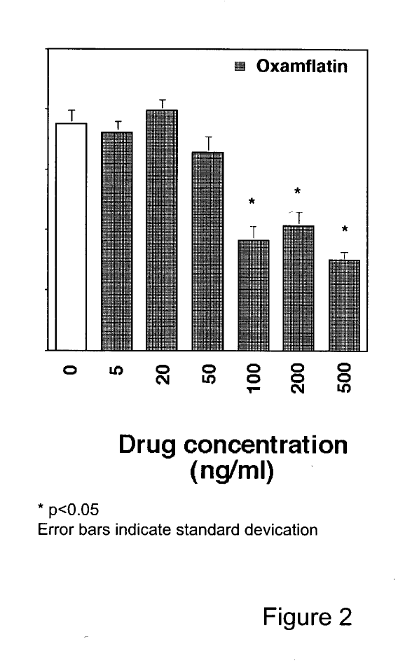

Referring to FIG. 2, micro-cultures of RASM cells were plated into a 96-well dish, and exposed as follows: a number of the micro-cultures were sham exposed, and a number were exposed to oxamflatin (5, 10, 50, 100, 200 and 500 ng/ml, respectively)., The RASM cells were exposed for 48 h before assessing cell proliferation.

### Example 5

### Testing Effests of HDAC-inhibitor-coated Stents in Vivo

The inventors' data show that HDAC inhibitors inhibit SMC proliferation in vitro. To confirm the effects demonstrated in non-cellular systems, the biological effects of stents coated with the active compound may be investigated in vivo by implanting stents into animal models, and monitoring the occurrence of restenosis after a period of time in the animal. Such tests confirm the utility of the methods of the invention for use in mammals, are useful for obtaining data on proper dosing of the drugs, and fulfill the mandate of the U.S. Food and Drug Administration for animal testing prior to use in human subjects. Specific guidelines outlining the required tests have recently been published (Schwartz et al., Drug-eluting stents in preclinical studies: recommended evaluation from a consensus group. Circulation, 106:1867-73, 2002).

Testing of the HDAC-inhibitor-coated stents may be carried out in pigs, which are commonly used for stent evaluation because of their anatomical (number and size of coronary arteries) and physiological (blood-clotting system) similarities to humans. The model described herein is the same previously used to test intra-coronary stents and drugs to prevent in-stent restenosis.

Juvenile, mongrel pigs of either sex, weighing 40-50 kg and in excellent health, may be used in the study. A 10-20% morality is typical for studies of this type in pigs, which are prone to ventricular fibrillation (VF) during manipulations of the coronary arteries, particularly if in-stent restenosis occurs. Therefore, bretylium may be infused to reduce the risk of VF, and the animals may be subjected to continuous ECG monitoring during the procedure. If VF does occur, it is treated with DC shock. The occurrence of VF after completion of the surgical procedure, when the pigs are not being monitored, will result in loss of the affected pigs.

This study incorporates formulations of HDAC inhibitors used on stents over periods of either 1 or 3 months:

stent+HDAC inhibitor for 1 month (n−2); and

stent+HDAC inhibitor for 3 months (n=10),

where n is the number of pigs tested. Therefore, stents are inserted in 20 pigs which, allowing for up to 20% mortality, ensures the survival of at least 16 pigs, which is the minimum number required for this preliminary study.

### Example 6

### Surgical Procedure Used to Test Efficacy of HDAC-Inhibitor-Coated Stents In Vivo

The following steps may be followed to test the efficacy of the HDAC-inhibitor-coated stents in vivo:

1. One day prior to the procedure, the animals are administered aspirin (325 mg/d PO) and ticlopidine (250 mg/d), to be continued daily until the animals are euthanized.

2. Ketamin (35 mg/kg) and glycopyrrolate (0.01 mg/kg) are administered intra-muscularly as premedication.

3. Intubation and placement of a venous line are performed.

4. The animals are anesthetized with inhaled isoflurane/oxygen.

5. The right femoral groin area is shaved and sterilized.

6. Cut-down to the right femoral artery or left carotid artery is carried out, followed by arteriotomy and insertion of an 8F introducer.

7. An in-dwelling subclavian catheter is inserted to withdraw blood samples.

8. For heparinization, a 10,000 U IV bolus is used, and the dosage is continued at 5000 U per hour.

9. Bretylium (5 mg/kg IV bolus) is administered, and then infusion is continued at 1 mg/min.

10. An 8F hockeystick coronary guiding catheter is passed retrograde over a 0.038″ guide wire to the aortic root, such that the left coronary artery will be engaged.

11. Intracoronary nitroglycerin (200 mcg) is administered.

12. Angiographic assessment of the left coronary artery is performed (by injection of the radiocontrast agent, 29% diatrizolate meglumine, during fluoroscopic visualization).

13. The angioplasty guide wire (High Torque Floppy) will be advanced into the LAD, and balloon angioplasty of the LAD will be performed (three 30-sec inflations at 8 ATM). Overstretch injury will be achieved using a balloon with a diameter 30% greater than the baseline arterial diameter.

14. After balloon removal, a 3.0-3.5 mm stent with coating appropriate for the experimental group will be implanted.

15. The angioplasty and stent procedures (steps 14-15) will be repeated for the LCX artery.

16. The hardware will be removed, the artery will be ligated, and the cut-down site will be closed in three layers, in a standard manner.

17. Animals will then be monitored for local bleeding and adequacy of limb perfusion in an intensive care unit for 24 h.

18. Two doses of cefazoline (25 mg/kg q 12 h) are administered after stent implantation.

19. Blood samples (10 ml each) are withdrawn at times zero, 48 h, 3 days, 1 week, 2 weeks, 3 weeks, and 4 weeks.

20. The animals are euthanized according to the AVMA Guidelines for Euthanasia (e.g., IV bolus injection of a cocktail of pentobarbital sodium, isopropyl alcohol, propylene glycol, and edetate sodium). Half of the animals are euthanized at 28 days, and the remaining half at 90 days post-procedure.

The pig hearts are then removed post-mortem, and the coronary arteries fixed with formalin. Histological evaluation for in-stent restenosis is then performed.

All materials used during the initial implantation are provided in a sterilized state, with appropriate labeling and documentation. All catheters and implants (stents) are used in single animals only. Basic surgical equipment used for cut-down is sterilized by the animal facility between uses. The surgical site is prepared and maintained in an aseptic condition, throughout the procedure, by shaving the right femoral groin or right neck, sterilizing the site by local application of polidine and alcohol (70%), and covering non-sterile areas with sterile drapers.

### Example 7

### Cell Migration Assay

This assay is more formally termed a “chemotaxis assay”, since it measures the number of cells that move through a porous membrane toward a chemoattractant (e.g., a chemical or growth factor) in a given period of time. Nevertheless, it may still be referred to as a “migration assay”.

Primary cells and cell culture media may be obtained from Clonetics (Walkersville, Md.), and grown at 37° C. with 5% CO₂. Primary human coronary artery smooth muscle cells (HCASMC) may be used at passage number≦10. The cells may be grown in smooth muscle cell basal medium (modified MCDB 131), with the addition of 5% fetal bovine serum (FBS), 0.5 tg/ml human epidermal growth factor (hEGF), 5 mg/ml insulin, 1.0 μg/ml human fibroblast growth factor, 50 mg/ml gentamycin, and 50 μg/ml amphoteracin B. Primary human coronary vascular endothelial cells (HCVEC) may be used at passage number≦10. The cells may be grown in endothelial cell basal medium (modified MCDB 131), with the addition of 5% FBS, 10 μg/ml hEGF, 1.0 mg/ml hydrocortisone, 3 mg/ml bovine brain extract, 50 mg/ml gentamycin, and 50 μg/ml amphoteracin B.

Cells may be removed from flasks by brief exposure to trypsin-EDTA (Invitrogen), followed by inactivation in complete medium, centrifugation for 5 min at 2,000 rpm, and resuspension in basal medium at a concentration of 2×10⁵ cells per 250 μl. Cells may be pipetted into the upper chamber of BD Falcon FluoroBlok™ 24-well insert plates (modified Boyden chambers; BD Biosciences, Bilerica, Mass.), containing fibronectin-coated filters, with either 3-μm or 8-μm pores. The lower chamber may contain basal medium with the addition of chemoattractants, such as serum or growth factors. For HCASMC, either FBS or human platelet derived growth factor BB (hPDGF-BB) may be used. For HCVEC, either FBS or human vascular endothelial growth factor (hVEGF) may be used.

After cells are added to the top chamber, along with various concentrations of drugs that are being tested for the inhibition of migration, the bottom chamber may be filled with 0.75 ml of basal medium containing chemoattractant. The plates may then be incubated for either 6 h or 22 h, at 37° C. At the end of the incubation period, liquid in the top chamber of each well may be aspirated, and the top half of the plate (containing the 24 upper chambers, to which the permeable filters are fused) may be lifted off, and excess liquid may be shaken into a sink. The top half of the plate may then be placed into a fresh 24-well plate, each well of which contains 0.75 ml Calcein AM solution (4 Vtg/ml; Molecular Probes, Eugene, Oreg.). The complete assembly may be incubated at 37° C. for 90 min, during which time the Calcein AM stains the cells that remained attached to the filter.

The stained plate may then be placed in a Victor II plate reader (PerkinElmer, Boston, Mass.) that is programmed to read from the bottom, with excitation at 485 nm, emission at 535 nm, and a 0.1 sec read time. Since the filter through which the cells have migrated has a dark, opaque color, the excitation or emission light does not penetrate the filter. Thus, only cells that have migrated through to the underside of the filter will be detected by the fluorescence plate reader. Data, recorded in arbitrary fluorescence units and analyzed using Prism v 3.02 (Graphpad Software), are typically expressed as percent migration.

### Example 8

### Proliferation Assay

This assay may be used to measure the number of live cells in a tissue culture dish or well. It does so by monitoring the color change of the tetrazolium salt, WST-1, which is modified by a mitochondrial enzyme involved in respiration. This enzyme is only active in living cells. The assay is similar to others, such as MTT or MTS, which measure the same activity using different tetrazolium chromophores.

Primary cells and cell culture media may be obtained from Clonetics (Walkersville, Md.), and grown at 37° C. in a humidified incubator containing 5% CO₂. Primary human coronary artery smooth muscle cells (HCASMC) may be used at passage number≦10. They may be grown in smooth muscle cell basal medium (modified MCDB 131) with the addition of: 5% fetal bovine serum (FBS); 0.5 μg/ml human epidermal growth factor (hEGF); 5 mg/ml insulin; 1.0 μg/ml human fibroblast growth factor; 50 mg/ml gentamycin; and 50 μg/ml amphoteracin B. Primary human coronary vascular endothelial cells (HCVEC) may be used at passage number≦10. They may be grown in endothelial cell basal medium (modified MCDB 131) with the addition of: 5% FBS; 10 μg/ml hEGF; 1.0 mg/ml hydrocortisone; 3 mg/ml bovine brain extract; 50 mg/ml gentamycin; and 50 μg/ml amphoteracin B.

Cells may be removed from flasks by brief exposure to trypsin-EDTA (Invitrogen), followed by inactivation in complete medium, centrifugation for 5′ at 2,000 rpm, and re-suspension in complete medium. Cells may be counted using a hemocytometer, and plated into 96-well tissue culture plates at 5×103 cells/well in 50 μl.

Test compounds may be dissolved in either DMSO or PBS, such that the final concentration of DMSO in the assay is 0.2%. Compounds may be prepared at twice the final assay concentration in complete medium, and 50 μl may be added to each well. The plates may then be incubated for 2-7 days, at 37° C. At the end of the incubation period, 10 μl WST-1 reagent (Roche Molecular Biochemicals, Indianapolis, Ind.) may be added to each well, followed by incubation at 37° C. for 90 min. During this time, the color change in the WST-1 reagent correlates with the number of live cells in each well. At the end of the incubation period, plates containing live cells may be analyzed immediately, or 15 μl of 10% sodium dodecyl sulfate (SDS) can be added to each well, thereby lysing the cells and preserving the assay for later analysis. Plates may be analyzed (0.1 sec/well) for absorbance at 450 nm in a Victor II plate reader (PerkinElmer, Boston, MA). Data may be expressed as arbitrary absorbance units (correlating with the number of live cells), and analyzed using Prism v 3.02 (Graphpad Software). Attorney Docket No. 5199-84

Discussed below are results obtained by the inventors in connection with the experiments of Example 1-4:

As shown in FIG. 1, exposure of RASM cells to trichostatin-A for 48 h significantly reduced light absorption, which indicates a significant inhibition of SMC proliferation (IC_{50 ˜}20 ng/ml). Additionally, inhibition of RASM cell proliferation by trichostatin-A was greater than that exhibited by rapamycin at equal concentrations of 50 ng/ml, thereby indicating that trichostatin-A is much more potent than rapamycin with regard to inhibition of SMC proliferation. As illustrated in FIG. 3, trichostatin-A produced similar results in HASM cells. Oxamflatin, an aromatic sulfonamide with HDAC inhibitory properties (IC₅₀ ˜75 ng/ml), also inhibited RASM cell proliferation, as can be seen in FIG. 2.

The experimental data derived by the inventors indicate that the two HDAC inhibitors, trichostatin-A and oxamflatin, potently inhibit rat and human vascular SMC proliferation, and trichostatin-A is much more potent than rapamycin at certain concentrations. Cell-viability assays using trypan blue exclusion demonstrated no significant difference after 48 h among control cells, RASM cells treated with rapamycin (100 nM), and RASM cells treated with trichostatin-A (100 ng/ml). This indicates that the HDAC inhibitors are not cytotoxic at the concentrations used in the Examples.

While the foregoing invention has been described in some detail for purposes of clarity and understanding, it will be appreciated by one skilled in the art, from a reading of the disclosure, that various changes in form and detail can be made without departing from the true scope of the invention in the appended claims.

What is claimed is:

1. A medical device for use in inhibiting proliferation and/or migration of smooth muscle cells, wherein the medical device has a coating comprising an HDAC inhibitor.

2. The medical device of claim 1, wherein the HDAC inhibitor is selected from the group consisting of trichostatin-A, suberoylanilide hydroxamic acid (SAHA), trapoxin, butyric acid, MS-27-275, oxamflatin, apicidin, depsipeptide, and depudecin.

3. The medical device of claim 1, wherein the medical device is a balloon catheter.

4. The medical device of claim 1, wherein the medical device is a stent for implantation in a blood vessel.

5. The medical device of claim 4, wherein the HDAC inhibitor is trichostatin-A or oxamflatin.

6. The medical device of claim 4, wherein the coating further comprises a biodegradable carrier that degrades over time, thereby allowing the HDAC inhibitor to elute from the stent over time.

7. The medical device of claim 6, wherein the coating further comprises a non-thrombogenic agent that is eluted from the stent as the biodegradable carrier degrades over time.

8. The medical device of claim 4, comprising a plurality of coatings, wherein each coating comprises a biodegradable carrier and at least one HDAC inhibitor that is eluted from the stent by staged release.

9. The medical device of claim 8, wherein at least one of the plurality of coatings further comprises an active ingredient that is eluted from the stent by timed release.

10. Use of a medical device in a method for inhibiting proliferation and/or migration of smooth muscle cells, wherein the medical device has a coating comprising an HDAC inhibitor.

11. A medical device for use in inhibiting proliferation and/or migration of smooth muscle cells, wherein the medical device has a coating comprising a biodegradable carrier that degrades over time and an HDAC inhibitor, and wherein the HDAC inhibitor is selected from the group consisting of trichostatin-A, suberoylanilide hydroxamic acid (SAHA), trapoxin, butyric acid, MS-27-275, oxamflatin, apicidin, depsipeptide, and depudecin.

12. The medical device of claim 11, wherein the medical device is a stent for implantation in a blood vessel.

13. The medical device of claim 12, wherein the HDAC inhibitor is trichostatin-A or oxamflatin.

14. Use of a medical device in a method for inhibiting proliferation and/or migration of smooth muscle cells, wherein the medical device has a coating comprising a biodegradable carrier that degrades over time and an HDAC inhibitor, and wherein the HDAC inhibitor is selected from the group consisting of trichostatin-A, suberoylanilide hydroxamic acid (SAHA), trapoxin, butyric acid, MS-27-275, oxamflatin, apicidin, depsipeptide, and depudecin.

15. A stent for implantation in a blood vessel, wherein the stent has a coating comprising a biodegradable carrier that degrades over time and an HDAC inhibitor, and wherein the HDAC inhibitor is trichostatin-A or oxamflatin.

16. A method for inhibiting proliferation and/or migration of non-neoplastic smooth muscle cells in a subject, comprising administering to the subject an amount of an HDAC inhibitor effective to inhibit proliferation of smooth muscle cells in the subject.

17. The method of claim 16, wherein the smooth muscle cells are vascular smooth muscle cells.

18. The method of claim 16, wherein the proliferation of smooth muscle cells is associated with a condition selected from the group consisting of stenosis, restenosis after angioplasty, restenosis after stent implantation, and accelerated arteriopathy after cardiac transplantation.

19. The method of claim 16, wherein the HDAC inhibitor is selected from the group consisting of trichostatin-A, suberoylanilide hydroxamic acid (SAHA), trapoxin, butyric acid, MS-27-275, oxamflatin, apicidin, depsipeptide, and depudecin.

20. The method of claim 16, wherein the HDAC inhibitor is coated on a medical device, and wherein the HDAC inhibitor is administered directly to the subject at a site susceptible to proliferation of smooth muscle cells.

21. The method of claim 20, wherein the medical device is a stent, and wherein the stent is implanted into the subject's vasculature.

22. The method of claim 17, wherein the stent is implanted into the subject's vasculature in a balloon angioplasty procedure.

23. A method for preventing or treating restenosis after angioplasty or stent implantation in a subject, comprising administering to the subject an amount of an HDAC inhibitor effective to prevent restenosis in the subject.

24. The method of claim 23, wherein the HDAC inhibitor is selected from the group consisting of trichostatin-A, suberoylanilide hydroxamic acid (SAHA), trapoxin, butyric acid, MS-27-275, oxamflatin, apicidin, depsipeptide, and depudecin.

25. The method of claim 23, wherein the HDAC inhibitor is trichostatin-A.

26. The method of claim 25, wherein the trichostatin-A is coated on a stent, and wherein the trichostatin-A is administered directly to the subject by implanting the stent into the subject at a site susceptible to restenosis.
